(19) United States
(12) Patent Application Publication
(10) Pub. No.: US 2006/0184080 A1
(43) Pub. Date: Aug. 17, 2006

(54) Reusable cervical collar having a removable larynx access opening cover

(76) Inventor: Albert Santelli JR., Middlesex, NJ (US)

Correspondence Address: DUANE MORRIS LLP, PO BOX 5203, PRINCETON, NJ, 08543-5203, US

(21) Appl. No.: 11/336,206

(22) Filed: Jan. 20, 2006

Related U.S. Application Data
(60) Provisional application No. 60/645,516, filed on Jan. 20, 2005.

(51) Int. Cl. A61F 5/00

(52) U.S. Cl. 602/18

(57) ABSTRACT

A cervical collar includes a collar member for encircling the neck of a patient in restrict movement of the head and neck of the patient and a removable closure disposed in a front portion of the collar member. The closure, when removed from the front portion of the collar member, provides an opening for accessing the larynx of the patient to perform a tracheotomy.

### RELATED APPLICATIONS

This application claims the benefit of U.S. Provisional Application No. 60/645,516, filed on Jan. 20, 2005, the entire disclosure of which is incorporated herein by reference.

Copending U.S. patent application Ser. No. 10/224,990, entitled REUSABLE CERVICAL COLLAR HAVING A CHIN STRAP MEMBER FASTENING ELEMENT WITH A PULL CORD, filed on Aug. 22, 2002 by Albert Santelli, Jr.

### FIELD OF THE INVENTION

This invention relates to cervical collars. More specifically, this invention relates to a reusable cervical collar having a larynx access openings.

### BACKGROUND OF THE INVENTION

There are many types of cervical collars that are designed to restrict movement of the head and neck of a person who has suffered a neck or spinal injury. More recent cervical collars are generally constructed from relatively stiff, light-weight plastic materials that are capable of being bent to encircle the neck of the injured patient and yet still provide substantial support for the patient.

Cervical collars commonly include an elongated neck encircling collar member and a strap-like chin support member attached to the collar member. The chin support member of the collar is located under the patient's chin after the collar member of the collar has been positioned around the neck of the patient. The chin support member is typically formed with a snap-fastening element that is snapped into a corresponding aperture in the collar member to secure the free end of the chin support member to the collar member. The other end of the chin support member is attached to the collar member during the manufacturing process.

It is sometimes necessary during an emergency medical situation to have access to a patient's larynx to perform a tracheotomy. For this reason, existing cervical collars are provided with an opening, which exposes the patient's larynx to permit a tracheotomy to be performed therethrough with the cervical collar in place. However, this opening weakens the collar and may allow the portion of the collar disposed under the chin to buckle and collapse from the weight of the patients head, thereby allowing the head of the patient to bend down abruptly, which may injure or further injure the patient

Accordingly, there is a need for an improved cervical collar that provides access to a patient's larynx to perform a tracheotomy, but which will not collapse.

### SUMMARY

A cervical collar is disclosed that comprises a collar member for encircling the neck of a patient to restrict movement of the head and neck of the patient and a removable closure disposed in a front portion of the collar member. The closure, when removed from the front portion of the collar member, provides entry to an opening in the collar for accessing the larynx of the patient to perform a tracheotomy.

### BRIEF DESCRIPTION OF THE DRAWINGS

FIG. 1 is a front elevational view of an embodiment of a cervical collar.

FIG. 2 is a rear elevational view of the cervical collar of FIG. 1.

FIG. 3A is an elevational view of a front portion of a collar member of the cervical collar of FIG. 1, which shows a plug-type closure for closing a tracheal access opening formed in the collar member front portion.

FIG. 3B is a partial sectional view showing the closure installed in the tracheal access opening.

FIG. 3C is a partial sectional view showing the closure removed from the tracheal access opening.

FIG. 4 is an elevational view of a front portion of the collar member of the cervical collar of FIG. 1, which shows a tear or knock-out type closure that, when removed, forms a tracheal access opening in the collar member front portion.

FIG. 5 is a partial sectional view showing another embodiment of the closure prior to removal from the collar member front portion.

FIG. 6 is a partial sectional view showing yet another embodiment of the closure prior to removal from the collar member front portion.

FIGS. 7-10 depict a method for assembling the chin strap member to the collar member wherein FIGS. 7 and 9 are front elevational views of the cervical collar and FIGS. 8 and 10 are rear elevational views of the cervical collar.

### DETAILED DESCRIPTION

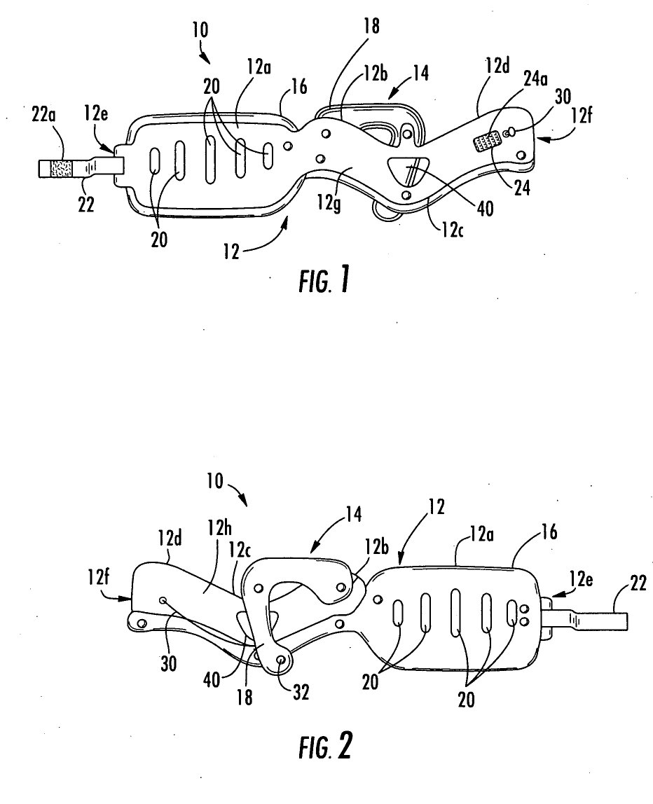

Referring collectively to FIGS. 1 and 2, there is shown an illustrative embodiment of a cervical collar 10. The collar 10 generally comprises an elongated collar member 12, that is capable of being rolled to encircle the neck of a patient, and a V-shape chin strap member 14, which supports the chin of the patient.

The collar member 12 may be constructed as a unitary, asymmetrical component comprised of a back portion 12a, a first side portion 12b, a U-shape front portion 12c, and a second side portion 12d. The back portion 12a defines collar first end 12e and the second side portion 12d defines collar second end 12f. The chin strap member 14 may be partially assembled to the collar member 12 as shown in FIGS. 1 and 2 to allow flat packaging and storage of the collar 10.

The collar and chin strap members 12 and 14 may be formed from somewhat rigid, plastic sheet material, such as high density polyethylene. The members may be die cut or injection molded as is conventional in the industry. Although the plastic sheet material is somewhat rigid, it is still flexible enough to be rolled so that when it is formed into the collar member 12, the collar member 12 can be placed around the patient's neck.

Still referring to FIGS. 1 and 2, a first pad element 16 may cover portions the inner surface 12h of the collar member 12 that contact the patient's body or head. A second pad element 18 may cover the inner surface of the chin strap member 14, and overlaps the edges of this member 14. The pad elements 16 and 18 prevent the edges and the inners surface portions of the collar and chin strap members from pressing and rubbing uncomfortably against the patient's body, head and neck. The pad elements 16 and 18 may be formed conventionally from foam using die cutting or the like.

A plurality of air holes 20 may extend through the back portion 12a and a corresponding portion of the pad element 16. A collar retaining strap 22, which may include a first element 22a of a hook and loop fastening system, may be provided on the back portion 12a of the collar member 12 and a strap retaining mechanism 24, which may include a corresponding second element 24a of the hook and loop fastening system, may be provided on the second side portion 12d of the collar member 12 for coupling the back portion 12a of the collar member 12 to the second side portion 12d of the collar member 12 to hold the cervical collar 10 securely in place around the neck of the patient. In one embodiment of use, the U-shape front portion 12c of the collar member 12, which has affixed to it a center portion of the chin strap member 14, may be located in the front of the patient's neck under the patient's chin, and the back portion 12a of the collar member 12 may be placed behind the back of the patient's neck.

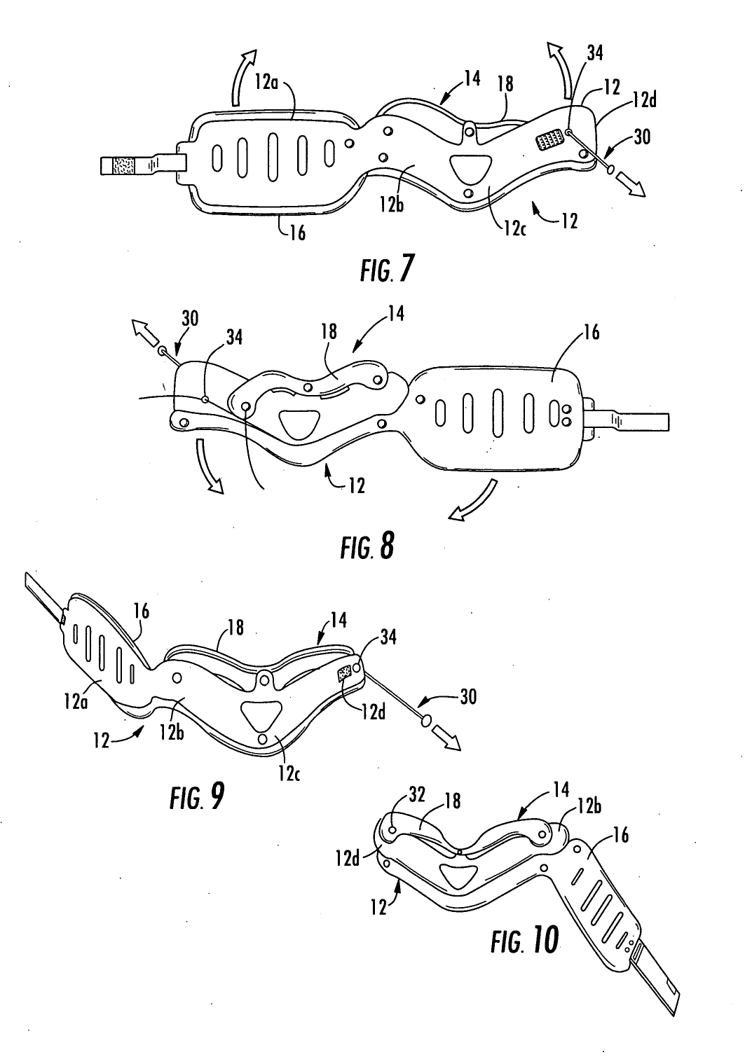

FIGS. 7-10 illustrate one method for assembling the chin strap member 14 to the collar member 12. A pull cord 30 of a fastening element 32 connected to the unattached end of the chin strap 14, is grasped and pulled to bend the chin strap member 14 and the front portion 12c of the collar member 12 into a curvilinear, operational shape. Continued pulling of the pull cord 30 moves the chin strap member 14 into position against the second side portion 12d of the collar member 12 and pulls the fastening element 32 into aperture 34 in the second side portion 12d of the collar member 12, thereby securing the chin strap member 14 to the collar member 12 and completing the assembly of the collar 10. The fully assembled collar 10 may then be placed around the patient's neck and secured with the collar retaining strap 22. Further details of this cervical collar and assembling method are described in copending U.S. patent application Ser. No. 10/224,990 filed on Aug. 22, 2002 by Albert Santelli, Jr., the inventor herein.

Referring again to FIGS. 1 and 2, the U-shape front portion 12c of the collar member 12 generally includes an access opening 40 which aligns with the larynx of a patient's neck when the cervical collar 10 is worn thereby, and permits a tracheotomy to be performed therethrough with the cervical collar 10 in place. A removable closure, as will be described in further detail immediately below, is provided for closing the access opening 40. When disposed in the access opening 40, the removable closure maintains the strength of the U-shape front portion 12c of the collar member 12, so that it will not buckle and collapse from the weight of the patient's head.

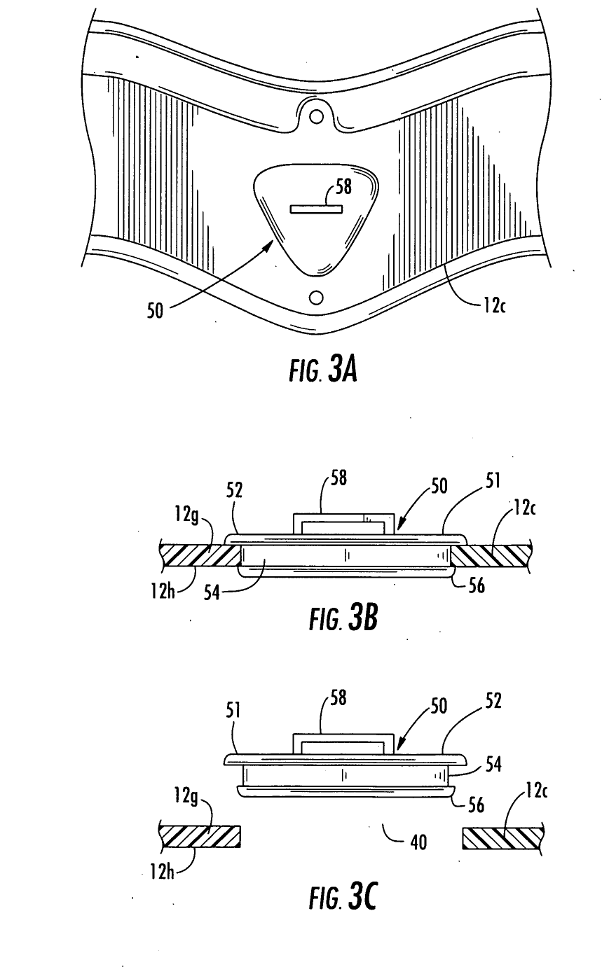

In some embodiments, as shown in FIGS. 3A-3C, the removable closure may comprise a type of closure 50 which may be securely reinstalled in the access opening 40, if desired. The closure 50 may be retained in the access opening 40 using any suitable retaining arrangement or structure. In the embodiment shown in FIGS. 3A-3C, the closure 50 may include a wall 51 surrounded by a flange 52 and a plug member 54 with a retaining bead 56. The flange 52 may be formed as a continuous or segmented element, and may be sized to substantially prevent the closure 50 from being pushed through the access opening 40. The plug member 54 extends from an inner surface of the wall 51 and be sized to extend through and plug the access opening 40. The retaining bead 56 may be provided at or adjacent to the free end of the plug member 54 to help retain the closure 50 within the access opening 40, while still allowing removal of the closure 50 from the access opening 40 when desired. The retaining bead 56 may be formed as a continuous or segmented element. A handle 58 may be provided on the outer surface of the closure 50 to facilitate removal thereof from the access opening 40. Other finger or tool grippable structures including, without limitation, pull cords, tabs, projections, may be used in place of the handle 58 for facilitating removal of the closure 50 from the access opening 40. The closure 50 may be unitarily formed of any material suitable for use in a collar member. In some embodiments, the closure 50 may be made of a polymeric material.

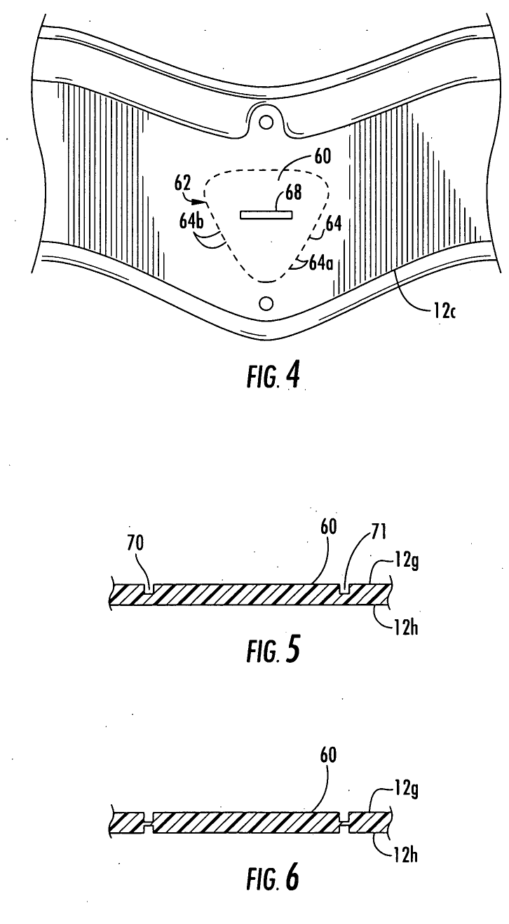

In other embodiments, as shown in FIG. 4, the closure may comprise a unitarily formed tear or knock-out type closure 60 formed in the front portion 12c of the collar member. The closure 60 may be made removable by providing a line of weakness 62 that circumscribes a selected area of the collar member front portion 12c which aligns with the larynx of the patient's neck when the cervical collar 10 is worn thereby. The line of weakness 63 allows the closure 60 to be torn out from the collar member front portion 12c, thereby forming the access opening 40. The line of weakness 63 may be embodied as a segmented line 64 comprising a plurality of perforations or cut lines 64a. The areas of the front portion 12c disposed between the ends of the cut lines or perforations 64a form frangible bridges 64b. The cut lines or perforations 64a may be made using any suitable cutting or perforating method. The closure 60 may be removing by pressing on the closure 60 with a force sufficient to fracture or tear the frangible bridges 64b. The opening left in the collar member front portion 12c by removing the closure 60 forms the earlier described access opening.

As shown in FIG. 5, the line of weakness may also be constructed as a groove 70 formed in the outer surface 12g or inner surface 12h of the collar member front portion 12c. The groove may be configured such that a frangible web 71 is formed at the bottom of the groove 70. The closure 60 may be removed by pressing on the closure 60 with a force sufficient to fracture or tear the web 71. The opening left in the collar member front portion 12c by removing the closure 60 forms the earlier described access opening.

FIG. 6 shows yet another embodiment of the line of weakness. In the embodiment of FIG. 6, the line weakness may be constructed as a first groove 80a formed in the outer surface 12g of the collar member front portion 12c and an oppositely disposed second groove 80b formed in the inner surface 12h of the collar member front portion 12c. The grooves 80a and 80b may be configured such that a frangible web 81 is formed between the first and second grooves 80a and 80b. The closure 60 may be removed by pressing on the closure 60 with a force sufficient to fracture or tear the web 81. The opening left in the collar member front portion 12c by removing the closure 60 forms the earlier described access opening.

A handle 68 (FIG. 4) may be provided on the outer surface of the closure 60 to facilitate removal of the closure 60 from the collar member front portion 12c. This feature is especially helpful when a tracheotomy must be performed sometime after the cervical collar has already been placed on the neck or a patient. Other finger or tool grippable structures including, without limitation, pull cords, tabs, projections, may be used in place of the handle 68 for facilitating removal of the closure 60.

While the foregoing invention has been described with reference to the above, various modifications and changes can be made without departing from the spirit of the invention. Accordingly, all such modifications and changes are considered to be within the scope of the appended claims.

What is claimed is:

1. A cervical collar comprising:
a collar member for encircling a neck of a patient to restrict movement of a head and the neck of the patient, the collar member having a front portion for positioning in front of a larynx of the neck of the patient; and
a removable closure disposed in the front portion of the collar member;
wherein removal of the closure from the front portion of the collar member provides entry to an opening in the front portion of the collar member for accessing the larynx of the patient.

2. The cervical collar according to claim 1, wherein the closure comprises a retaining structure which allows the closure to be reinstalled in the opening.

3. The cervical collar according to claim 2, further comprising a gripping structure for facilitating removal of the closure from the opening.

4. The cervical collar according to claim 1, wherein the closure comprises a unitarily formed section of the portion of the collar member.

5. The cervical collar according to claim 4, wherein the section is defined by a line of weakness.

6. The cervical collar according to claim 5, wherein the line of weakness comprises a segmented line.

7. The cervical collar according to claim 6, wherein the segmented line includes one of a plurality of perforations and a plurality of cut lines.

8. The cervical collar according to claim 7, wherein the segmented line includes a plurality of frangible elements disposed between the one of a plurality of perforations and a plurality of cut lines.

9. The cervical collar according to claim 6, wherein the segmented line includes a plurality of frangible elements.

10. The cervical collar according to claim 5, wherein the line of weakness comprises a groove formed in a surface of the portion of the collar member.

11. The cervical collar according to claim 10, wherein the line of weakness further comprises a frangible web disposed at a bottom of the groove.

12. The cervical collar according to claim 5, wherein the line of weakness further comprises a first groove formed in a first surface of the portion of the collar member and a second groove formed in a second surface of the portion of the collar member, opposite to the first groove.

13. The cervical collar according to claim 12, wherein the line of weakness further comprises a frangible web disposed between the first and second grooves.
